(19) United States
(12) Patent Application Publication
(10) Pub. No.: US 2026/0090832 A1
(43) Pub. Date: Apr. 2, 2026

(54) CALIBRATION BLOCK FOR SURGICAL FIDUCIAL MARKER ARRAYS

(71) Applicant: Medivis, Inc., New York, NY (US)

(72) Inventors: Jesse Harrison Gallant, New York, NY (US); Osamah Choudhry, New York, NY (US); Christopher Morley, New York, NY (US); Florentin Jonas Liebmann, New York, NY (US)

(21) Appl. No.: 18/900,842

(22) Filed: Sep. 29, 2024

(51) Int. Cl. A61B 17/88; A61B 90/00; A61B 17/00

(52) U.S. Cl. CPC A61B 17/8886; A61B 90/39; A61B 2017/00725

(57) ABSTRACT

A calibration block configured to facilitate the calibration of separate surgical fiducial marker arrays on separate medical devices includes a main body having a top face and multiple side faces, top surgical fiducial markers at the top face, side surgical fiducial markers at the side faces, and screw openings at the top face. The top face includes a reference divot configured to accept therein a separate surgical probe tip for separate fiducial marker array calibration. The top and side surgical fiducial markers combine to form a fixed asymmetrical positional arrangement of surgical fiducial markers for calibration. Each screw opening is configured to accept therein a separate surgical screw coupled to a separate surgical screwdriver having a separate surgical screwdriver fiducial marker array coupled thereto for calibration.

### TECHNICAL FIELD

The present disclosure relates generally to medical devices, and more particularly to tools and devices used during medical procedures.

### BACKGROUND

Planning and navigation are necessary for many medical procedures, such as live surgeries, practices, training, planning, scanning, and the like. Surgical teams typically have a plan based on medical imagery before ever entering an operating room. Conventional medical imaging systems such as X-ray, MRI, CT, and others have limitations regarding two-dimensional and three-dimensional images, however, and surgeons often need to consider numerous image views and slices to plan surgical procedures. Recent medical advances leverage these applications of medical imagery and surgical plans by using a computer-aided augmented reality environment, which can allow for the tracking of patients and physical instruments during surgical procedures by using reference (i.e., fiducial) markers and associated tracking components. Other medical procedures, such as ultrasound scanning, have also made use of computer-aided augmented reality environments involving tracked reference markers.

Unfortunately, conventional tracking systems are often limited in their ability to accurately generate, render, and apply virtual interactions in an augmented reality environment based on the orientations and positions of physical instruments with respect to those of physical landmarks identified on a patient body or other relevant location, particularly when things move during surgery or another relevant medical procedure. Unstable or unreliable positioning of fiducial markers can play a role in these issues. Limited or inaccurate tracking can then affect the overall performance of such systems during surgeries or other relevant medical procedures, and the need for accuracy in this regard can lead to overly cumbersome or complex attachment devices and systems.

To facilitate accurate tracking within an augmented reality environment, it is often necessary to calibrate the exact locations of fiducial markers relative to a physical instrument or other item being used with the fiducial markers, such as a surgical fiducial marker array having multiple surgical fiducial markers that is coupled to the physical instrument or other item to be tracked. Known calibration techniques and procedures can often be cumbersome or repetitive, however, particularly when recalibration is needed for any adjustment of the fiducial markers or the removal or addition of a relevant component or item from a physical instrument being tracked. Furthermore, some physical instruments and other devices are not known to have ways of calibrating fiducial markers relative thereto, which then limits flexibility with respect to being able to adjust or reposition fiducial marker arrays for such items.

While traditional ways of virtually tracking items during surgery or other medical procedures have worked well in the past, improvements are always helpful. In particular, what is desired are systems and methods that provide more robust and streamlined ways to facilitate the calibration of fiducial marker locations relative to a physical instrument or other item being tracked during medical procedures in a simple and streamlined manner.

### SUMMARY

It is an advantage of the present disclosure to provide systems and methods that provide more robust and streamlined ways to facilitate the calibration of fiducial marker locations relative to a physical instrument or other item being tracked during medical procedures in a simple and streamlined manner. The disclosed features, apparatuses, systems, and methods relate to calibration devices that can be used to facilitate the calibration of fiducial markers prior to or during surgeries and other medical procedures. In particular, the disclosed systems and methods can involve a calibration device, such as a calibration block, that can be specifically configured to facilitate the calibration of a surgical fiducial marker array coupled to a surgical instrument or other item to be tracked. This calibration block or device can be configured to facilitate calibration for multiple different types of surgical instruments or other items, including those that involve the removal of surgical screws or other components, without requiring recalibration upon the addition of new surgical screws or other components.

In various embodiments of the present disclosure, a calibration device configured to facilitate calibration of surgical fiducial markers can include at least a main body, a plurality of primary fiducial marker couplers, and a plurality of secondary fiducial marker couplers. The main body can have a primary face and a plurality of secondary faces oriented in directions that are different than the orientation of the primary face. The primary face can include at least one reference divot configured to accept therein the tip of a separate medical device having a plurality of separate surgical fiducial markers coupled thereto. The plurality of primary fiducial marker couplers can be located at the primary face, and each of the plurality of primary fiducial marker couplers can being configured to couple a surgical fiducial marker thereto. The plurality of secondary fiducial marker couplers can be located at two or more of the plurality of secondary faces, and each of the plurality of secondary fiducial marker couplers can also be configured to couple a surgical fiducial marker thereto. The plurality of primary fiducial marker couplers and plurality of secondary fiducial marker couplers can combine to form a fixed asymmetrical positional arrangement for surgical fiducial markers configured to be referenced by a separate augmented reality system for calibrating the separate surgical fiducial markers.

In various detailed embodiments, the plurality of separate surgical fiducial markers can be arranged on a separate surgical fiducial marker array. The separate medical device can be a surgical probe, such as a pedicle probe. The calibration device can also include surgical fiducial markers coupled at each of the plurality of primary fiducial marker couplers and each of the plurality of secondary fiducial marker couplers. The primary face can be a top face of the calibration device and the plurality of secondary faces can be side faces of the calibration device. At least one of the plurality of secondary faces having secondary fiducial marker couplers can also include at least one reference divot configured to accept therein the tip of the separate medical device having the separate surgical fiducial markers coupled thereto.

In further detailed embodiments, the primary face can also include one or more screw openings. Each of the one or more screw openings can be configured to accept therein a separate surgical screw coupled to a separate surgical screwdriver having a separate surgical screwdriver fiducial marker array coupled thereto. Each of the one or more screw openings can be configured to facilitate calibration of the separate surgical screwdriver fiducial marker array while the separate surgical screw is fully accepted therein and coupled to the separate surgical screwdriver. In some arrangements, calibration of the separate surgical screwdriver fiducial marker array does not need to be repeated when the separate surgical screw is detached from the separate surgical screwdriver and another separate surgical screw is coupled thereto. Each of the one or more screw openings can include a bottom surface that matches the end surface of the separate surgical screw. The one or more screw openings can include multiple screw openings of different diameters, each of the different diameters corresponding to a standard diameter size for a surgical screw. Each of the multiple screw openings can have the same depth. In some arrangements, the separate surgical screw can be a pedicle screw and the separate surgical screwdriver can be a pedicle screwdriver.

In various further embodiments of the present disclosure, a calibration block configured to facilitate the calibration of surgical fiducial marker arrays on separate medical devices can include a main body, a plurality of top surgical fiducial markers, a plurality of side surgical fiducial markers, and a plurality of screw openings. The main body can have a top face oriented upwards and multiple side faces oriented sideways. The top face can include a reference divot configured to accept therein the tip of a separate surgical probe having a separate surgical probe fiducial marker array coupled thereto. The plurality of top surgical fiducial markers can be located at the top face, and the plurality of side surgical fiducial markers can be located at two or more of the multiple side faces. The plurality of top fiducial markers and the plurality of side fiducial markers can combine to form a fixed asymmetrical positional arrangement of surgical fiducial markers configured to be referenced by a separate augmented reality system for calibrating the separate surgical fiducial marker array. The plurality of screw openings can be located at the top face, and each of the plurality of screw openings can be configured to accept therein a separate surgical screw coupled to a separate surgical screwdriver having a separate surgical screwdriver fiducial marker array coupled thereto.

In various detailed embodiments, the separate surgical probe can be a pedicle probe and the separate surgical screwdriver can be a pedicle screwdriver. Each of the plurality of screw openings can have a different diameter, and each of the different diameters can correspond to a standard diameter size for a surgical screw. Each of the plurality of screw openings can have a depth of about 45 mm. Two or more of the multiple side faces having side surgical fiducial markers can also include a reference divot configured to accept therein the tip of the separate surgical probe coupled with the separate surgical probe fiducial marker array.

In still further embodiments of the present disclosure, various methods of using a calibration device are provided. Pertinent process steps can include coupling a first surgical screw to the tip of a surgical screwdriver having a surgical fiducial marker array coupled thereto, inserting the first surgical screw into a screw hole on a primary face of the calibration device while the first surgical screw is coupled to the tip of the surgical screwdriver, and calibrating the locations of a plurality of surgical screwdriver fiducial markers on the surgical fiducial marker array. Calibration can be done using an augmented reality calibration system that references a plurality of primary surgical fiducial markers located on the primary face of the calibration device and a plurality of secondary surgical fiducial markers located on one or more secondary faces of the calibration device.

In various detailed embodiments, additional process steps can include coupling a medical device chuck and the surgical fiducial marker array to the surgical screwdriver, selecting the first surgical screw based on desired screw dimensions, selecting the screw hole from a plurality of screw holes located on the primary face of the calibration device to match the first surgical screw, inputting the selected screw hole into the augmented reality calibration system, performing a first medical operation that detaches the first surgical screw from the surgical screwdriver, coupling a second surgical screw to the tip of the surgical screwdriver, and performing a second medical operation that detaches the second surgical screw from the surgical screwdriver without recalibrating the surgical fiducial marker array. Additional screws can be coupled and detached in subsequent medical operations as may be desired.

Other apparatuses, methods, features, and advantages of the disclosure will be or will become apparent to one with skill in the art upon examination of the following figures and detailed description. It is intended that all such additional apparatuses, methods, features and advantages be included within this description, be within the scope of the disclosure, and be protected by the accompanying claims.

### BRIEF DESCRIPTION OF THE DRAWINGS

The included drawings are for illustrative purposes and serve only to provide examples of possible structures, arrangements, and methods of use for calibration blocks and other calibration devices for calibrating surgical fiducial marker arrays. These drawings in no way limit any changes in form and detail that may be made to the disclosure by one skilled in the art without departing from the spirit and scope of the disclosure.

FIG. 1 illustrates in front perspective view an example calibration block configured to facilitate the calibration of surgical fiducial marker arrays according to one embodiment of the present disclosure.

FIG. 2A illustrates in front perspective view an example alternative calibration block according to one embodiment of the present disclosure.

FIG. 2B illustrates in bottom perspective view the calibration block of FIG. 2A according to one embodiment of the present disclosure.

FIG. 2C illustrates in top plan view the calibration block of FIG. 2A according to one embodiment of the present disclosure.

FIG. 2D illustrates in closeup partial front perspective view the calibration block of FIG. 2A with a surgical probe tip referenced at a primary face surface divot thereof according to one embodiment of the present disclosure.

FIG. 2E illustrates in full front perspective view the calibration block and surgical probe arrangement of FIG. 2D according to one embodiment of the present disclosure.

FIG. 3A illustrates in side perspective view an example surgical screw.

FIG. 3B illustrates in side perspective view an example surgical screwdriver.

FIG. 3C illustrates in side perspective view an example system of a surgical screwdriver and associated surgical fiducial marker array coupled to a surgical screw according to one embodiment of the present disclosure.

FIG. 4 illustrates in top perspective view the system of FIG. 3C with its coupled surgical screw fully inserted into a screw opening of the calibration block of FIG. 2A according to one embodiment of the present disclosure.

FIG. 5 illustrates a flowchart of an example summary method of using a calibration device according to one embodiment of the present disclosure.

FIG. 6 illustrates in closeup partial top perspective view an example surgical screw prior to insertion into a screw opening of the calibration block of FIG. 2A according to one embodiment of the present disclosure.

FIG. 7A illustrates in side elevation view an example surgical screw prior to insertion into a screw opening of the calibration block of FIG. 2A according to one embodiment of the present disclosure.

FIG. 7B illustrates in side elevation view an example surgical screw fully inserted into a screw opening of the calibration block of FIG. 2A according to one embodiment of the present disclosure.

FIG. 8 illustrates a flowchart of an example detailed method of using a calibration block according to one embodiment of the present disclosure.

### DETAILED DESCRIPTION

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

As is generally well known, modern surgeries and other medical procedures are sometimes facilitated by using a computer-aided augmented reality environment. Fiducial markers can be used for tracking patients, medical devices, or other physical instruments during surgeries or other medical procedures. Such fiducial markers can be part of overall systems that can include specialized lighting arrangements, cameras, and computing systems. Attachment devices are often used to locate the fiducial markers in place relative to the patient or other physical device or instrument. To facilitate accurate tracking within an augmented reality environment, it is often necessary to calibrate the exact locations of fiducial markers relative to a physical instrument or other item being used. Known calibration techniques and procedures can often be cumbersome or repetitive, particularly when recalibration is needed for any adjustment of the fiducial markers or the removal or addition of a relevant component or item from a physical instrument being tracked. Furthermore, calibration procedures are simply not done for certain types of medical instruments, such as surgical screwdrivers, for example.

The disclosed calibration blocks provide more robust and streamlined ways to facilitate the calibration of fiducial marker locations relative to a physical instrument or other item being tracked during medical procedures, and also provide ways to calibrate fiducial marker locations with respect to alternative instruments, such as surgical screwdrivers. In particular, a disclosed calibration block or device can be configured to facilitate calibration for multiple different types of surgical instruments or other items, including those that involve the removal of surgical screws or other components, without requiring recalibration upon the addition of new surgical screws or other components.

In various detailed embodiments of the present disclosure, a novel calibration block or device can be configured to calibrate surgical fiducial marker arrays with respect to surgical probes and also to surgical screwdrivers with surgical screws coupled thereto, among other possible instruments and items. This can involve the presence of multiple fiducial markers and multiple reference surface divots on multiple different faces of the calibration block, as well as a plurality of differently sized screw openings on a primary face of the calibration block, among other components and features. Use of the disclosed calibration blocks and methods can involve pedicle screw insertion procedures, for example, among other possible medical procedures.

Although various embodiments disclosed herein discuss calibration blocks configured to facilitate calibration of fiducial marker arrays coupled to pedicle probes and to pedicle screwdrivers for use in augmented reality aided pedicle probe and pedicle screw implantation procedures, it will be readily appreciated that the disclosed features, apparatuses, systems, and methods can also be used in conjunction with other devices and equipment that can leverage various advantages the disclosed calibration blocks and devices. It is thus specifically contemplated that other surgical probes, surgical screwdrivers, and alternative applications with other medical devices may also apply. For example, the disclosed calibration blocks can be used to facilitate calibration of surgical clamps having fiducial marker arrays coupled thereto and a reference pointer adapted for placement into a calibration reference divot. Other applications, arrangements, and extrapolations beyond the illustrated embodiments are also contemplated.

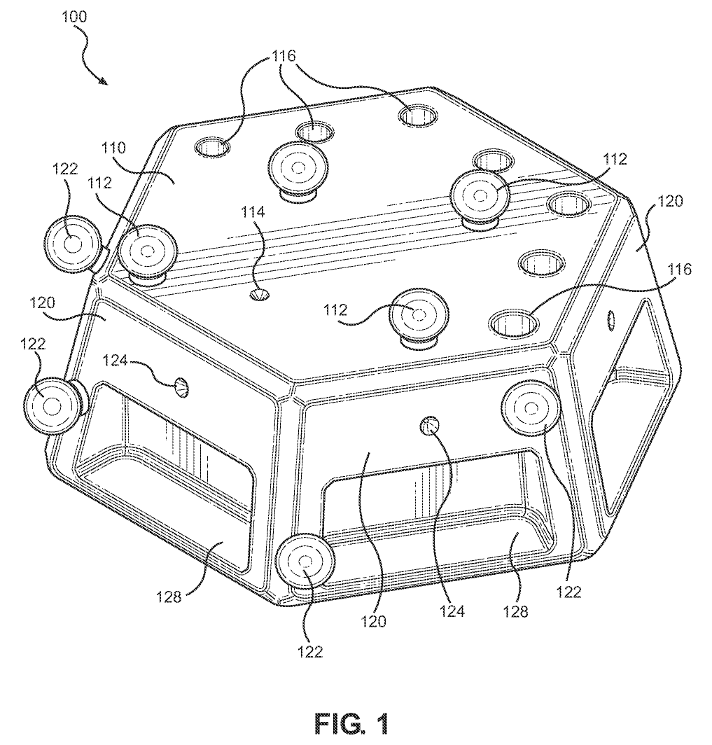

Referring first to FIG. 1, an example calibration block configured to facilitate the calibration of surgical fiducial marker arrays is illustrated in front perspective view. In general, calibration block 100 can be configured to facilitate the calibration of surgical fiducial marker arrays coupled to surgical probes, surgical screwdrivers, and other relevant medical devices. Calibration block 100, which can also be called a calibration device, can include a main body having a primary face 110 and a plurality of secondary faces 120 that can be oriented in directions that are different than the orientation of the primary face. Primary face 110 and each of secondary faces 120 can be generally planar with various features on or about its surface. As shown, primary face 110 can be a top face oriented upwards, while secondary faces 120 can be side faces oriented sideways with respect to the top face. In some arrangements, calibration block 100 can form a hexagonal shape having six secondary faces 120, although other shapes, configurations, and face orientations are also possible.

Primary face 110 can include a plurality of primary fiducial markers 112 (i.e., surgical fiducial markers) coupled thereto, and these primary fiducial markers can generally form an asymmetrical pattern about the primary face, as shown. One or more of secondary faces 120 can include a plurality of secondary fiducial markers 122 coupled thereto, and these can be the same as or substantially similar to primary fiducial markers 112. Primary and secondary fiducial markers 112, 122, which can be top and side surgical fiducial markers as shown, can be infrared reflective spheres, retroreflective spheres, infrared-emitting diodes, or any other suitable form of fiducial marker, as are generally well known. Primary and secondary fiducial markers 112, 122 can combine to form a fixed asymmetrical positional arrangement of surgical fiducial markers configured to be referenced by a separate augmented reality system for calibrating the locations of separate surgical fiducial markers, as will be understood by those of skill in the art.

Primary face 110 can also include a primary reference divot 114 on its surface, and this primary reference divot can be configured to accept therein the tip of a separate medical device, such as a surgical probe. This separate surgical probe, which can be a pedicle probe, for example, can have its tip inserted into primary reference divot 114 such that the locations of a separate set of fiducial markers coupled to the surgical probe can then be calibrated using this primary reference divot and some or all of primary and secondary fiducial markers 112, 122. The separate set of fiducial markers can be arranged on a separate surgical fiducial marker array coupled to the separate surgical probe.

One or more of secondary faces 120 can include one or more secondary reference divots 124 on their surfaces, and these secondary reference divots can similarly be configured to accept therein the tip of a separate surgical probe or other medical device. Accordingly, a similar calibration process can be conducted with respect to any of these secondary reference divots 124 and some or all of primary and secondary fiducial markers 112, 122. It will be appreciated that a calibration process can use a reference divot 114 or 124 and some or all of the primary and secondary fiducial markers 112, 122 arranged at known relative locations to locate the separate fiducial markers in three-dimensional space relative to the tip of the surgical probe such that an augmented reality assisted environment can be provided for subsequent us of the surgical probe. One or more of secondary faces 120 can also include an indentation or recess 128, and these features can be used to handle calibration block 100 or to affix the calibration block in place for a calibration procedure, such as to a table or shelf.

Primary face 110 can also include one or more screw openings 116 located at its surface. For example, primary face 110 can have seven different screw openings 116 distributed around its surface as shown, although fewer or more screw openings can be used. Each screw opening 116 can be configured to accept therein a separate surgical screw, such as a pedicle screw, for example. The separate surgical screw can be coupled to a separate surgical screwdriver having a separate surgical screwdriver fiducial marker array coupled thereto when the surgical screw is inserted into a screw opening 116, whereupon the screwdriver fiducial marker array can then be calibrated, as set forth in greater detail below.

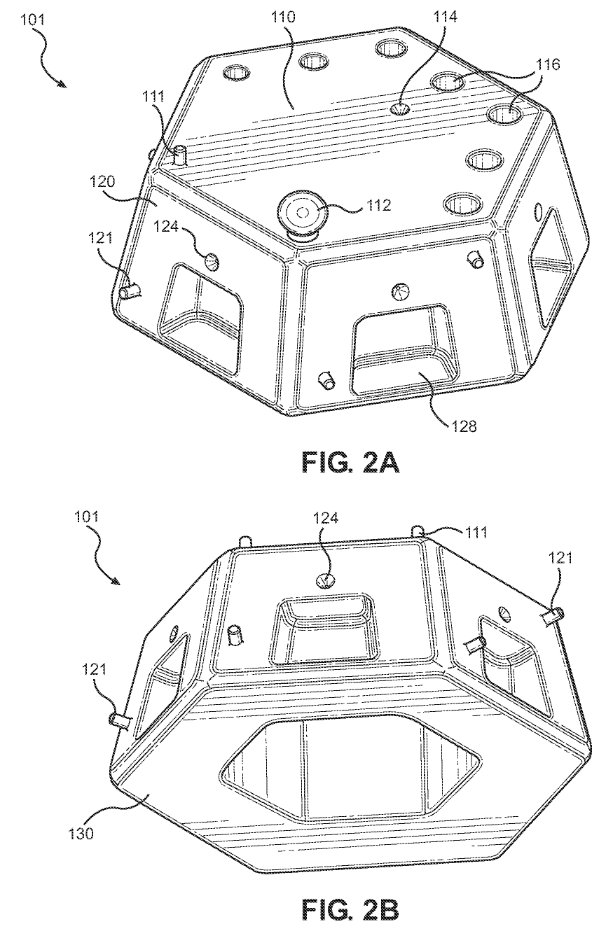

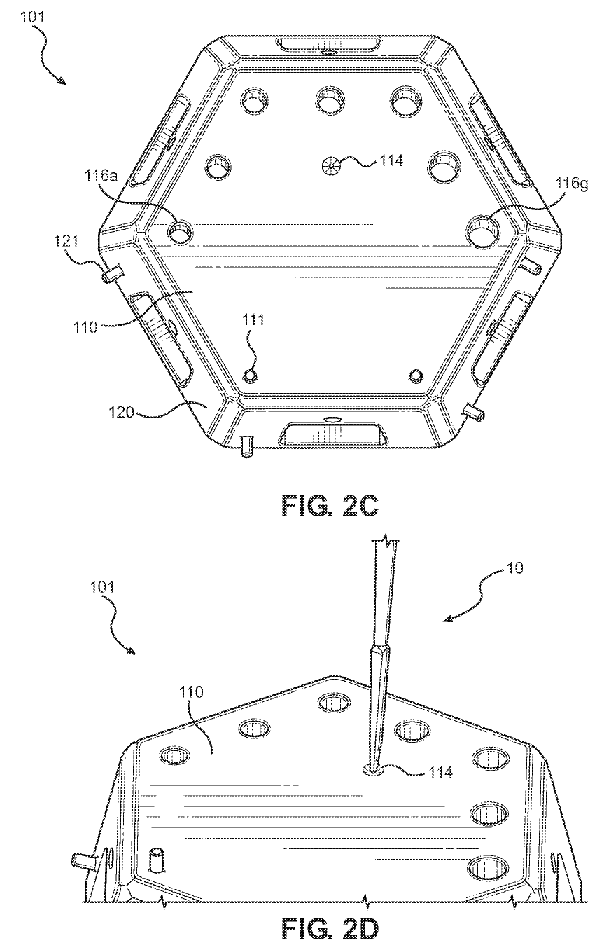

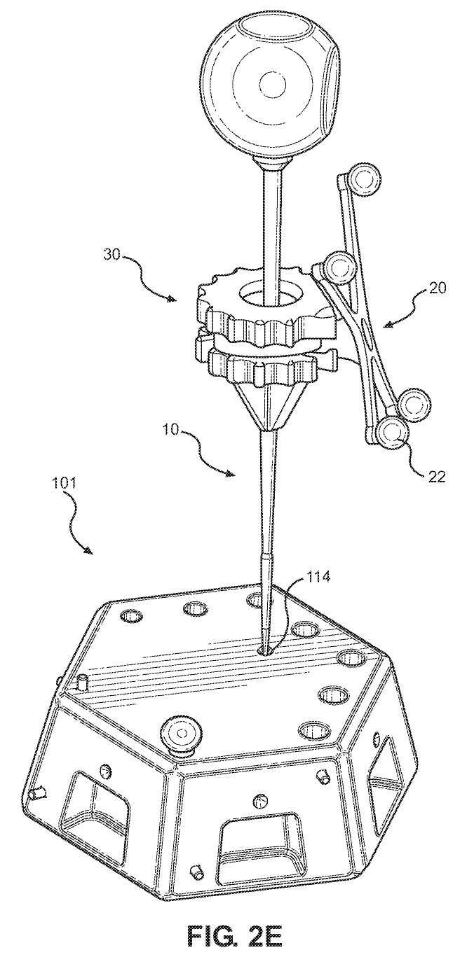

Moving next to FIGS. 2A through 2C, an example alternative calibration block is shown in front perspective, bottom perspective, and top plan views respectively. Calibration block 101 can be substantially similar to calibration block 100 above except for a few different design details. For example, top face 110 includes a different arrangement of primary fiducial markers 112 and a different location for primary reference divot 114 relative to other calibration block features. It will be readily appreciated that a variety of different amounts, arrangements, and configurations for primary and secondary fiducial markers 112, 122, primary and secondary reference divots 114, 124, and screw openings 116 can be designed without detracting from the overall functionalities of a calibration block.

In various embodiments, some or all of the primary and secondary fiducial markers 112, 122 can be removably coupled to calibration block 101. As such, calibration block 101 is shown as having fiducial marker couplers 111 and 121 at primary and secondary faces 110, 120 respectively. These fiducial marker couplers 111, 121 can be coupling posts or features extending from the primary and secondary faces at designated locations where removable fiducial markers are to be coupled. It will be understood that fiducial marker couplers 111, 121 and removable fiducial markers can similarly be used for calibration block 100 above as well. As shown in FIG. 2B, bottom surface 130 of calibration block 101 can include a central opening or other features suitable for mounting or affixing the calibration block to a table or other firm supporting location. As shown in FIG. 2C, calibration block 101 can also have seven screw openings 116a-116g distributed around primary face 110.

Continuing with FIGS. 2D and 2E, an example calibration block is shown with a surgical probe tip referenced at a primary face divot in closeup and full front perspective views respectively. FIG. 2D simply depicts a closeup view of the tip of a separate surgical probe 10 being inserted into primary reference divot 114 on primary face 110 of calibration block 100. FIG. 2E provides greater context and shows a full arrangement including a pedicle probe 10 and a surgical fiducial marker array 20 with four fiducial markers 22 coupled to a medical device chuck 30 that is removably clamped onto the pedicle probe. Pedicle probe 10 is shown as being situated with its tip in primary reference divot 114 such that its fiducial markers 22 can be calibrated. Details regarding an arrangement of such a pedicle probe 10, surgical fiducial marker array 20, and medical device chuck 30 can be found in related U.S. Patent Application No. __/___,___ entitled “MEDICAL DEVICE CHUCK WITH ROTATING FIDUCIAL MARKER ARRAY,” which application is hereby incorporated by reference in its entirety.

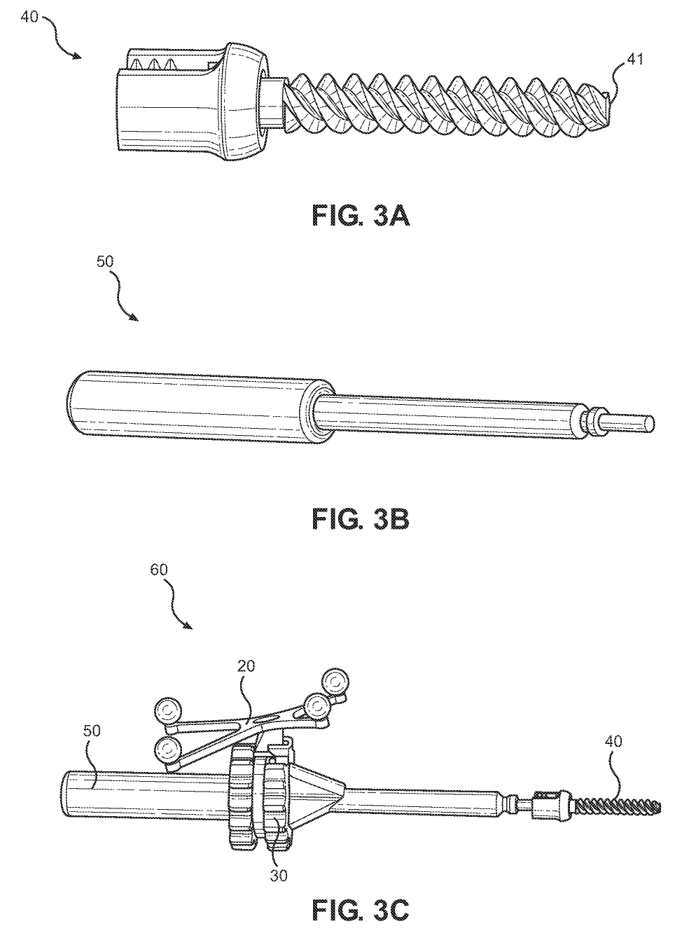

Transitioning now to FIGS. 3A-3C, an alternative medical device suitable for calibration by the disclosed calibration blocks will now be discussed. FIG. 3A illustrates in side perspective view an example surgical screw. Surgical screw 40 can be a typical pedicle screw, for example, with a coupling element configured to removably coupled to a pedicle screwdriver and a flattened front end 41. FIG. 3B illustrates in side perspective view an example surgical screwdriver. Surgical screwdriver 50 can be configured to couple at its front end to the coupling portion of surgical screw 40 in a manner such that the surgical screw can be inserted into a patient and then detached from the surgical screwdriver. FIG. 3C illustrates in side perspective view an example system of a surgical screwdriver and associated surgical fiducial marker array coupled to a surgical screw. Surgical system 60 can include the surgical screw 40 coupled to the surgical screwdriver 50 as shown, as well as a surgical fiducial marker array 20 coupled to the surgical screwdriver by way of medical device chuck 30. Surgical system 60 can be used during a medical operation to insert surgical screw 40 into a patient using an augmented reality system that tracks the fiducial markers of surgical fiducial marker array 20, whereupon the surgical screw can then be detached from surgical screwdriver 50. Again, this can involve insertion of a pedicle screw using a pedicle screwdriver, for example.

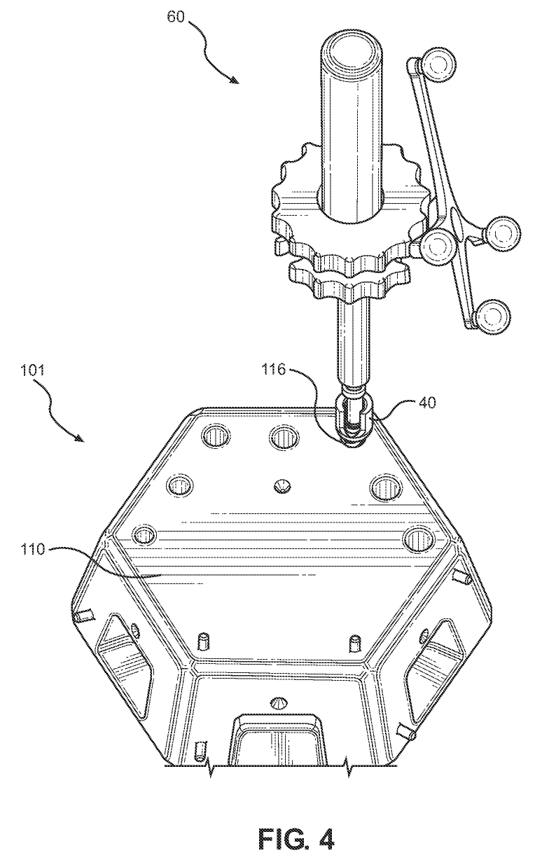

Next, FIG. 4 illustrates in top perspective view the surgical system of FIG. 3C with its coupled surgical screw fully inserted into a screw opening of the calibration block of FIG. 2A. Surgical system 60 can be arranged vertically with respect to calibration block 101 such that surgical screw 40 is fully inserted into a suitable screw opening 116 on primary face 110 of the calibration block. In various embodiments, each of screw openings 116 can be of a different diameter such that a snug fit within a suitable screw opening can be made for any of a variety of different sized surgical screws. In some embodiments, the depths of the screw openings 116 can vary with respect to each other as may be desired. Alternatively, each screw opening 116 can be of the same depth, such as 45 mm, for example. As shown, surgical system 60 is positioned such that calibration of its surgical fiducial marker array can take place using calibration block 101.

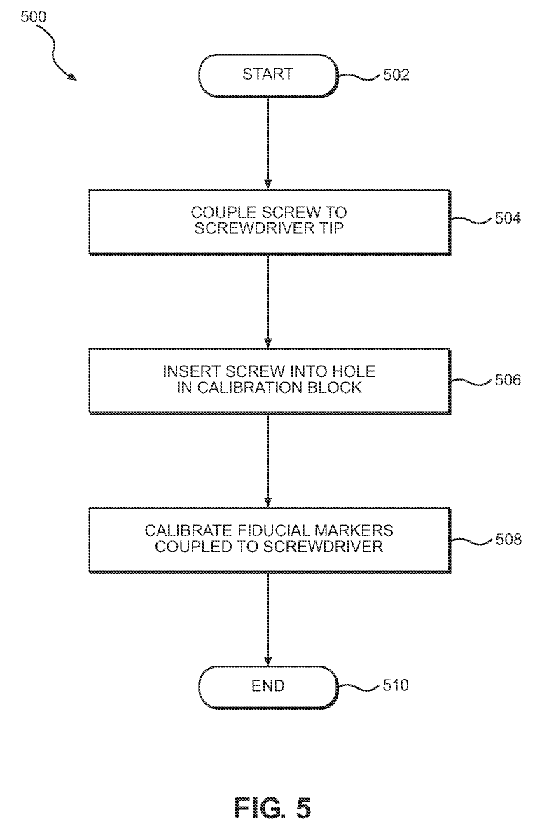

Turning now to FIG. 5, a flowchart of an example summary method of using a calibration device is provided. Summary method 500 can represent one broad aspect for overall methods of use for a calibration block, and it will be understood that various other steps, features, and details of such a broad aspect and overall methods of use are not provided here for purposes of simplicity. While the disclosed calibration block can be designed for use with a pedicle probe, pedicle screwdriver, or other surgical probe or screwdriver as disclosed herein, it is contemplated that the calibration block can also be used with other objects and in other environments and applications.

After a start step 502, a first process step 504 can involve coupling a surgical screw to the tip of surgical screwdriver. This can involve standard surgical screw and screwdriver equipment with these items designed and made to couple together, as is generally well known. The surgical screwdriver can have a surgical fiducial marker array coupled thereto, as detailed above. Step 504 can be performed manually or automatically, such as where a separate robotic system can be configured to handle the surgical screw and surgical screwdriver.

At the following process step 506, the surgical screw can be inserted into a screw hole or opening on a calibration block while the surgical screw is coupled to the surgical screwdriver. The screw opening can be on a primary face of the calibration block. These items can be those illustrated and described in detail above, and the screw opening can be a suitably sized opening designed to accommodate the surgical screw coupled to the surgical screwdriver. Step 506 can be manually or automatically performed, such as where a separate robotic system can be configured to handle the surgical screw, surgical screwdriver, and calibration block.

At subsequent process step 508, the locations of surgical screwdriver fiducial markers on an array coupled to the surgical screwdriver can be calibrated. This can be done using an augmented reality calibration system that references a plurality of primary surgical fiducial markers located on the primary face of the calibration block or device and a plurality of secondary surgical fiducial markers located on one or more secondary faces of the calibration block or device. Step 508 can be manually or automatically performed, such as where software of an augmented reality calibration system can be i to calibrate the marker locations based on input and detected parameters. The method can then end at end step 510.

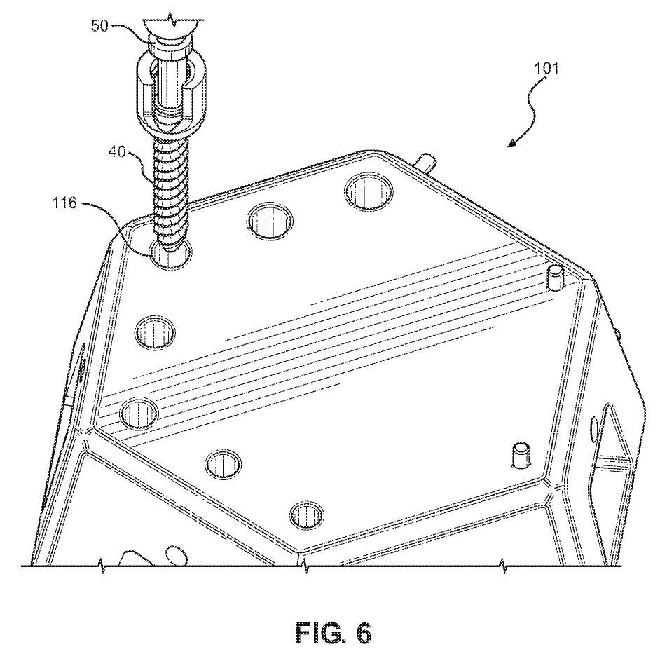

FIG. 6 illustrates in closeup partial top perspective view an example surgical screw prior to insertion into a screw opening of the calibration block of FIG. 2A. As shown, surgical screwdriver 50 is positioned such that the surgical screw 40 coupled to a front end thereof is just about to be inserted into a suitable screw opening 116 in calibration block 101. Again, the suitable screw opening 116 can be selected such that the opening diameter matches the diameter of the surgical screw 40 so as to provide a snug fit between the opening and the screw.

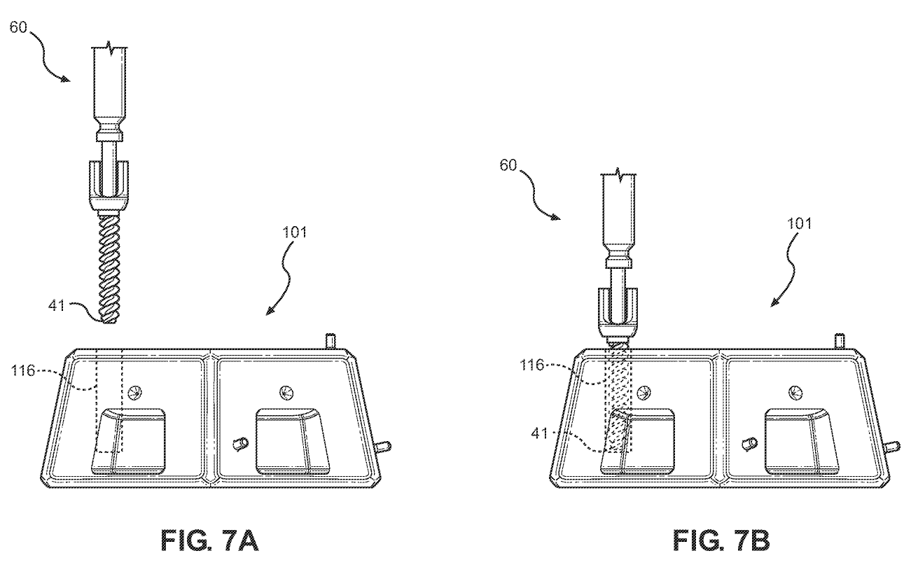

Next, FIGS. 7A and 7B illustrate in side elevation views an example surgical screw prior to insertion into a screw opening of a calibration block and the same surgical screw fully inserted into the screw opening respectively. FIG. 7A provides an alternative view to that which is shown in FIG. 6, with the surgical screw of surgical system 60 about to be inserted into screw opening 116 of calibration block 101. Flat front end 41 of the screw can be inserted in screw opening 116 until it abuts against the bottom floor of the screw opening, as shown in FIG. 7B. At this point, calibration of the fiducial markers on surgical system 60 can take place given the known length of surgical screw 40, the known depth of screw opening 116, the known location of the screw opening, and the known locations of the fiducial markers on calibration block 101.

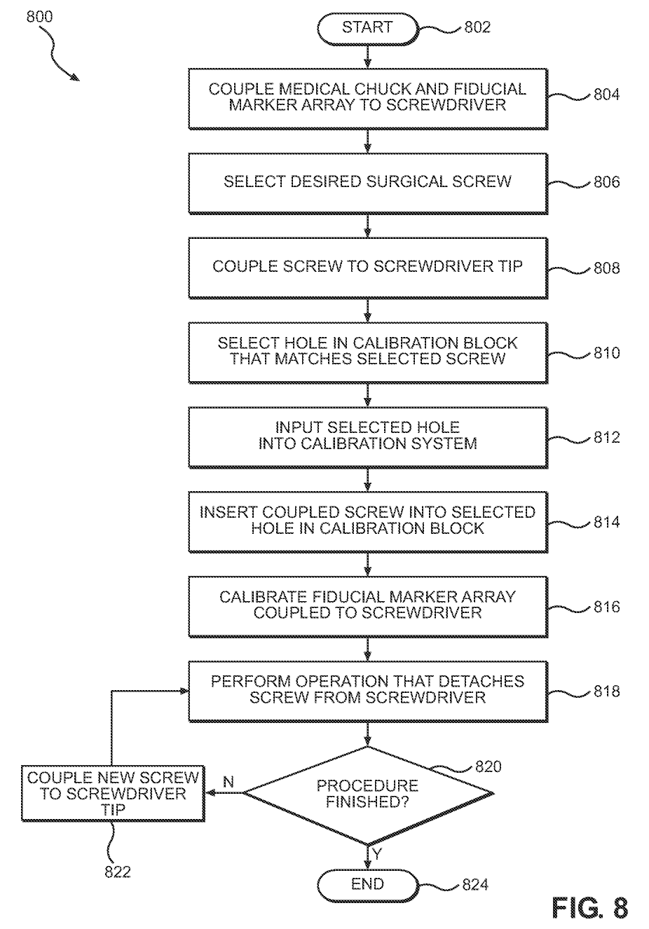

Lastly, FIG. 8 illustrates a flowchart of an example detailed method of using a calibration block. Detailed method 800 can represent one possible way of using a calibration block, and it will be understood that various other steps, features, and details of such a detailed method are not provided here for purposes of simplicity. Detailed method 800 can also include some or all steps and details of summary method 500 above, as will be readily appreciated. After a start step 802, a first process step 804 can involve coupling a medical device chuck and surgical fiducial marker array to a surgical screwdriver to be used for a medical procedure to implant surgical screws in a given patient. Step 804 can be optional in some situations, such as where a medical system is available with these devices already coupled together. Step 804 can be manually or automatically performed, such as where a separate robotic system can be configured to handle and couple together the medical device chuck, the surgical fiducial marker array, and the surgical screwdriver.

A following process step 806 can involve selecting a desired surgical screw. This can involve choosing a screw to be used on a given patient based on factors specific to that patient and what is needed. For example, a small pedicle screw might be selected based on a small patient or the inability of the relevant patient pedicle to be able to accommodate or support a larger pedicle screw. Step 806 can be performed manually or automatically in some cases, such as where software of a medical system can be configured to select an appropriate surgical screw based on pertinent parameters and data specific to a given patient.

The next process step 808 can be identical or substantially similar to step 504 above, and can involve coupling the selected surgical screw to the tip of surgical screwdriver. This can involve standard surgical screw and screwdriver equipment with these items designed and made to couple together, as is generally well known. The surgical screwdriver can have a surgical fiducial marker array coupled thereto, as detailed above. Step 808 can be performed manually or automatically, such as where a separate robotic system can be configured to handle the surgical screw and surgical screwdriver.

Subsequent process step 810 can involve selecting a proper screw opening or hole in the primary face of the calibration block based on the selected surgical screw. Again, the calibration block can have a plurality of screw openings, each of which can have a different diameter and some of which may have different depths or other features. The screw opening that best matches or corresponds to the surgical screw being used can be the one that is selected. Step 810 can be performed manually or automatically, such as where software of an augmented reality calibration system can be configured to select the best screw opening based on the surgical screw that is selected and any other pertinent parameters.

At a following process step 812, the selected screw opening or hole can be input into the calibration system. This can involve inputting the selected screw opening and any other variable parameters into the system so that the system can then use that information to be able to accurately calibrate the coupled fiducial markers with the known information. Step 812 can be manually or automatically performed, such as where software of an augmented reality calibration system can be configured to accept and process variable parameter inputs.

At the next process step 814, which can be identical or substantially similar to step 506 above, the selected surgical screw can be inserted into a screw hole or opening on a calibration block while the surgical screw is coupled to the surgical screwdriver. The screw opening can be on a primary face of the calibration block. These items can be those illustrated and described in detail above, and the screw opening can be a suitably sized opening designed to accommodate the surgical screw coupled to the surgical screwdriver. Step 814 can be manually or automatically performed, such as where a separate robotic system can be configured to handle the surgical screw, surgical screwdriver, and calibration block.

At the next process step 816, which can be identical or substantially similar to step 508 above, the locations of surgical screwdriver fiducial markers on an array coupled to the surgical screwdriver can be calibrated. This can be done using an augmented reality calibration system that references a plurality of primary surgical fiducial markers located on the primary face of the calibration block or device and a plurality of secondary surgical fiducial markers located on one or more secondary faces of the calibration block or device. Step 816 can be manually or automatically performed, such as where software of an augmented reality calibration system can be configured to calibrate marker locations based on inputs and detected parameters.

At process step 818, an operation can be performed that detaches the surgical screw from the surgical screwdriver. This can involve implanting the surgical screw inside a patient. For example, the operation can be inserting a pedicle screw into a patient pedicle with a pedicle screwdriver and then detaching the screw to implant it in the pedicle. Step 818 can be manually or automatically performed, such as where a separate robotic system can be configured to handle and operate the surgical screwdriver to perform the desired operation.

At the following decision step 820, an inquiry can be made as to whether the procedure is finished or if any further surgical screws are to be implanted. If the procedure is finished, then the method can move to end step 824. If more surgical screws are desired, however, then the method can proceed to process step 822, where a new surgical screw can be coupled to the tip of the surgical screwdriver. After that, the method can revert to and repeat process step 818 with the new surgical screw. Of note, no further calibration needs to take place for the coupling and detachment of any surgical screws after calibration has taken place with the first surgical screw. The method can then end at end step 824.

For foregoing method 800, it will be appreciated that not all process steps are necessary, and that other process steps may be added in some arrangements. For example, step 806 can be repeated where multiple surgical screws of different sizes or dimensions are to be used. Furthermore, the order of steps may be altered in some cases, and some steps may be performed simultaneously. For example, steps 810 and 812 may be performed before step 808. As another example, steps 810 and 812 can be performed simultaneously. Although known process steps are provided for the various techniques in detailed method 800, it will be appreciated that any other suitable similar method for using a calibration block can also be used. Other variations and extrapolations of the disclosed methods will also be readily appreciated by those of skill in the art.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims.

What is claimed is:

1. A calibration device configured to facilitate calibration of surgical fiducial markers, the calibration device comprising:
a main body having a primary face and a plurality of secondary faces oriented in directions that are different than the orientation of the primary face, wherein the primary face includes at least one reference divot configured to accept therein the tip of a separate medical device having a plurality of separate surgical fiducial markers coupled thereto;
a plurality of primary fiducial marker couplers located at the primary face, each of the plurality of primary fiducial marker couplers being configured to couple a surgical fiducial marker thereto; and
a plurality of secondary fiducial marker couplers located at two or more of the plurality of secondary faces, each of the plurality of secondary fiducial marker couplers being configured to couple a surgical fiducial marker thereto, wherein the plurality of primary fiducial marker couplers and plurality of secondary fiducial marker couplers combine to form a fixed asymmetrical positional arrangement for surgical fiducial markers configured to be referenced by a separate augmented reality system for calibrating the separate surgical fiducial markers.

2. The calibration device of claim 1, wherein the plurality of separate surgical fiducial markers are arranged on a separate surgical fiducial marker array.

3. The calibration device of claim 1, wherein the separate medical device is a surgical probe.

4. The calibration device of claim 1, further comprising:
surgical fiducial markers coupled at each of the plurality of primary fiducial marker couplers and each of the plurality of secondary fiducial marker couplers.

5. The calibration device of claim 1, wherein the primary face is a top face of the calibration device and the plurality of secondary faces are side faces of the calibration device.

6. The calibration device of claim 1, wherein at least one of the plurality of secondary faces having secondary fiducial marker couplers also includes at least one reference divot configured to accept therein the tip of the separate medical device having the separate surgical fiducial markers coupled thereto.

7. The calibration device of claim 1, wherein the primary face further includes one or more screw openings, each of the one or more screw openings being configured to accept therein a separate surgical screw coupled to a separate surgical screwdriver having a separate surgical screwdriver fiducial marker array coupled thereto.

8. The calibration device of claim 7, wherein each of the one or more screw openings is configured to facilitate calibration of the separate surgical screwdriver fiducial marker array while the separate surgical screw is fully accepted therein and coupled to the separate surgical screwdriver.

9. The calibration device of claim 8, wherein calibration of the separate surgical screwdriver fiducial marker array does not need to be repeated when the separate surgical screw is detached from the separate surgical screwdriver and another separate surgical screw is coupled thereto.

10. The calibration device of claim 7, wherein each of the one or more screw openings includes a bottom surface that matches the end surface of the separate surgical screw.

11. The calibration device of claim 7, wherein the one or more screw openings include multiple screw openings of different diameters, each of the different diameters corresponding to a standard diameter size for a surgical screw.

12. The calibration device of claim 11, wherein each of the multiple screw openings have the same depth.

13. The calibration device of claim 7, wherein the separate surgical screw is a pedicle screw and the separate surgical screwdriver is a pedicle screwdriver.

14. A calibration block configured to facilitate the calibration of surgical fiducial marker arrays on separate medical devices, the calibration block comprising:
a main body having a top face oriented upwards and multiple side faces oriented sideways, wherein the top face includes a reference divot configured to accept therein the tip of a separate surgical probe having a separate surgical probe fiducial marker array coupled thereto;
a plurality of top surgical fiducial markers located at the top face;
a plurality of side surgical fiducial markers located at two or more of the multiple side faces, wherein the plurality of top fiducial markers and the plurality of side fiducial markers combine to form a fixed asymmetrical positional arrangement of surgical fiducial markers configured to be referenced by a separate augmented reality system for calibrating the separate surgical fiducial marker array; and
a plurality of screw openings located at the top face, wherein each of the plurality of screw openings is configured to accept therein a separate surgical screw coupled to a separate surgical screwdriver having a separate surgical screwdriver fiducial marker array coupled thereto.

15. The calibration block of claim 14, wherein the separate surgical probe is a pedicle probe and the separate surgical screwdriver is a pedicle screwdriver.

16. The calibration block of claim 14, wherein each of the plurality of screw openings has a different diameter, each of the different diameters corresponding to a standard diameter size for a surgical screw.

17. The calibration block of claim 14, wherein each of the plurality of screw openings has a depth of about 45 mm.

18. The calibration block of claim 14, wherein two or more of the multiple side faces having side surgical fiducial markers also include a reference divot configured to accept therein the tip of the separate surgical probe coupled with the separate surgical probe fiducial marker array.

19. A method of using a calibration device, the method comprising:
coupling a first surgical screw to the tip of a surgical screwdriver having a surgical fiducial marker array coupled thereto;
inserting the first surgical screw into a screw hole on a primary face of the calibration device while the first surgical screw is coupled to the tip of the surgical screwdriver; and
calibrating the locations of a plurality of surgical screwdriver fiducial markers on the surgical fiducial marker array using an augmented reality calibration system that references a plurality of primary surgical fiducial markers located on the primary face of the calibration device and a plurality of secondary surgical fiducial markers located on one or more secondary faces of the calibration device.

20. The method of claim 19, further comprising the steps of:
coupling a medical device chuck and the surgical fiducial marker array to the surgical screwdriver;
selecting the first surgical screw based on desired screw dimensions;
selecting the screw hole from a plurality of screw holes located on the primary face of the calibration device to match the first surgical screw;
inputting the selected screw hole into the augmented reality calibration system;
performing a first medical operation that detaches the first surgical screw from the surgical screwdriver;
coupling a second surgical screw to the tip of the surgical screwdriver; and
performing a second medical operation that detaches the second surgical screw from the surgical screwdriver without recalibrating the surgical fiducial marker array.
